(19) United States
(12) Patent Application Publication
(10) Pub. No.: US 2004/0015233 A1
(43) Pub. Date: Jan. 22, 2004

(54) Cardiac valve prosthesis, especially mitral cardiac valve and method for producing the same

(76) Inventor: Josef Jansen, Koln (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/398,470

(22) PCT Filed: Oct. 2, 2001
(86) PCT No.: PCT/DE01/03810

(30) Foreign Application Priority Data
Oct. 9, 2000 (DE) 10050092.7

(51) Int. Cl. A61F 2/24

(52) U.S. Cl. 623/2.18; 623/2.38; 623/2.42

(57) ABSTRACT

The invention relates to a cardiac valve prosthesis, comprising a support housing with at least two flaps, especially to a mitral cardiac valve. The flaps and/or the support housing have a core and a surface layer enclosing said core, the core material being characterized by a greater hardness and/or lesser flexural elasticity than the surface layer. For producing the cardiac valve according to the invention the inner surface layers of the flaps and the support body are produced as an integral part by at least one dip-coating step in a liquid solution. A support body core is then injection-molded onto said structure. In further dip-coating steps the flap core zones are formed and the outer surface layers of the flaps and the support body are finally produced in at least one further dip-coating step and the body so produced is then removed from the dip mold.

The invention relates to a cardiac valve prosthesis comprised of a support housing with at least two cusps, especially a mitral heart valve.

From WO 97/49355, among other things, mitral heart valves have become known which are comprised of a support housing with a base ring which has two posts extending substantially in the axial direction of the ring and forming arcuate walls serving for the attachment of two flexible cusps, the posts having free ends which form internal seats for the cusps.

The cusps of such a mitral valve are, on physiological grounds, substantially flatter by comparison with aortic valve cusps and are formed with a significantly smaller radius of curvature. The stiffness of thus configured mitral cusps is therefore smaller than the stiffness in the case of aortic cusps.

Since, however, the pressure load in the mitral position is greater than that for the cusps of an aortic cardiac valve, they are therefore more strongly loaded. Basically, there is, however, the possibility to increase the thickness of the cusps but this gives rise to relatively high bending elongation on the surface. The consequences thereof can be different. Thus there is the danger that the cusps can come loose from the walls of the support housing or that the cusp flexibility at the connecting locations can become fatigued. Homogeneously soft thicker cusps also have the drawback that high bending forces are required to open the cusps or that the cusps will not open sufficiently. It also cannot be excluded that the cusps will tear along the commissure lines and/or that the cusp material will fatigue with time so that because of the corresponding material fatigue variations in the contours will occur and deposits will easily form on the cusps which will increase the general tendency toward thromboses. Similarly there is an increase in the tendency toward calcification since the lime preferably deposits at the regions of higher elongation.

To overcome the aforementioned disadvantage, it is proposed in U.S. Pat. No. 4,222,126 to reinforce the commissure lines of the cusp with a small elastomeric band and provide additionally reinforcing support ribs. It has, however, been found that the disadvantages mentioned at the outset are not sufficiently alleviated in this manner.

It is, therefore, an object of the present invention to provide a valve prosthesis, especially a mitral valve prosthesis, whose construction is improved from the point of view of durability.

This object is achieved with a cardiac valve prosthesis according to claim 1 in that the cusps and/or the support housing have a core and a surface coating enveloping this core, whereby the core material has a greater hardness and a reduced bending yield strength than the surface layer. Preferably the hardness and/or the bending yield strength in the support housing and/or the cusps vary from the outer lying region to the inwardly lying (core) regions gradually and with increasing penetration depth. In other words the core of the cusps (or of the support housing) is comprised of a material with a lesser tension-elastic property, that is a harder material, while the cover surfaces are formed from a biocompatible blood tolerable and clearly softer and more bendable material. In this manner the yield limits of the cusps can be significantly increased. Ideally this transition with increasing penetration depth is continuous. Through this feature, the reverse bending strength of the cusps is increased since softer material as a rule, especially when they are in the same polymer family, preferably polyurethanes, have higher yieldability [elongation, flexibility]. It is also known that harder materials, like, for example, polyurethanes with higher hard segment proportions tend to be less blood tolerable and to have lower creep rates than softer materials. Preferably materials are used for the sandwich like construction according to the invention which have the following module uses of elasticity, namely, for the outwardy lying surface area 4 to 40 N/mm², for the core of the cusps 40 to 200 N/mm² and for the stent material 200 to 1000 N/mm².

According to a further configuration of the invention, the core region of the cusp, which has a homogeneous material structure, has a thickness of 0.05 mm to 0.15 mm while the surface layer has a thickness of 0.02 mm to 0.1 mm so that the total thickness preferably amounts to 0.2 mm to 0.25 mm.

To protect the free cusp edge against crack formation and to increase simultaneously the sealing effectiveness of the closed cusp, the cusp edge zones which come to lie against one another upon closing of the cusps are configured as sealing lips with an edge-side thickening of the material of the surface coating, whereby the mutually abutting surfaces, considered in the throughflow direction, have a height of at least 0.35 mm, preferably from 0.5 mm to 0.6 mm. With the distribution of the cusp in a core region and a softer surface zone with a sealing lip embossment at the commissure end, the cusp is protected on the one hand against a breakdown in an effective manner and on the other hand the cusp edges are configured to be uniformly flexible and elastic so that the durability against repeated reverse bending of the cusp is enhanced which is an especially important advantage for the opening and closing movements.

Advantageously, the support housing and the cusps are composed of the same material, especially polyurethane, which have different mechanical properties in the core region and in the surface coatings. By contrast with such cardiac valve prostheses as utilize different materials for the support housing and the cusps, chemical interactions at the interfaces can be avoided.

To the extent a further stabilization of the base ring is desired, this can be achieved by inserting into the base ring of titanium or a titanium alloy. This ring is completely enveloped with the remaining material of the support housing, for example, with polyurethane.

The titanium or its alloys are largely inert chemically with respect to polyurethane and usually in the region of the base ring a sufficient thickness of polyurethane is provided for shielding the titanium ring or the zones adjoining same toward the exterior. In this manner, the ntire cardiac valve prosthesis can be completely formed from polyurethane.

The support housing itself or the core of the support housing in case this is comprised of a core and an edge structure, has a greater hardness and/or a lesser bending yield strength than the core of the cusps. With this feature account is taken that the flexibility and elasticity of the cusps must be greater than that of the support housing, especially also in the region of the posts.

To produce the described heart valve, preferably the production of the cusps in an immersion process is effected at the outset, whereby on an immersion core body of steel or a synthetic resin with polished surfaces, whose shape corresponds to the configuration of the cusp, initially in a plurality of immersion steps interrupted by respective drying steps, surface layers are produced. Then by an injection molding a support body core is cast, after which in further immersion steps, the cusp core region is formed and then finally by at least one further immersion process, the outer surface layers of the cusp and support body are applied before the so formed body is removed from the immersion mold.

According to a further development of the invention, the process according to the invention is so modified that at least one of the layers or a core layer is so formed that individual drops of a polymer solution or drops of a viscous polymerizable multicomponent system are applied in a point like manner in a row along a line, in a bead like manner or over a surfac ar a to the carrying tool or to a previously produced layer, the applied material is dried and application of the droplets and the subsequent drying is repeated as often as required until the desired layer is built up in a corresponding three dimensional configuration. An exact conformation of the individual droplets to the tool or the substrate product by an immersion process to which the droplets are to be applied, can be accomplished by a guided positioning device for a metering tool which is located at a space from the tool or substrate on which the desired layer is to be deposited and which is moved along by a triggering. The droplets can be deposited next to one another so that they come into contact and in total form a continuous, optionally also liquid polymer film. Thus can several or many layers be built up in succession to a defined thickness of the foil, for example, in such form that in the production of the cusps, the free cusp edges are configured with a (thicker) sealing lip. Alternatively, thereto, it is possible to deposit noncontacting droplets and after drying to fill in the interstices with them with new droplets so as to produce a grid forming pattern of the desired film in a corresponding thickness. The volumetric flow supplied by the metering system is comprised of reproducible individual droplets whose size in diameter is 0.2 mm to 1 mm corresponding to a volume of 34 nl to 4.2 μl. The flattened diameter of the applied droplets is preferably 0.25 mm to 2.5 mm. Ideally a polymer solution is coupled by dropwise application optimally when the viscosity of the polymer solution used amounts to 1 mPas to 50 Pas.

The aforedescribed metering process can also be combined with casting and immersion processes in accordance with the state of the art, for example such that on a core body the cusps are produced by alternating immersion in a polymer solution and the metered application of individual droplets to form the respective layers. Here as well a plurality of respective immersion or metering processes are required. After the separation of the free cusp edges, by casting or corresponding further immersion processes and/or metered droplet application the stent body can be formed onto them, whereby between the individual immersion, casting or metering stages, a metal ring, which preferably consists of titanium or a titanium alloy, can be shoved on and in further processes coated and enclosed with the desired polymer, especially polyurethane.

Exemplary embodiments of the invention are illustrated in the drawings. They show:

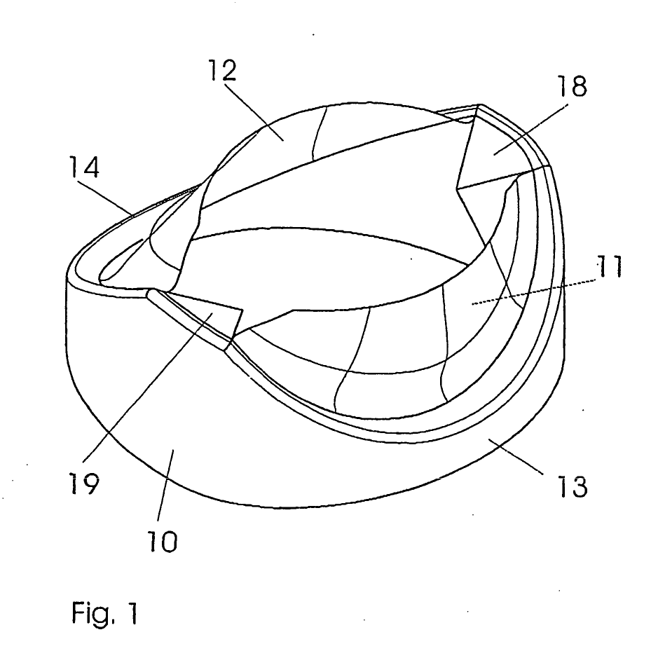

FIG. 1 is a perspective view of a prosthetic mitral heart valve;

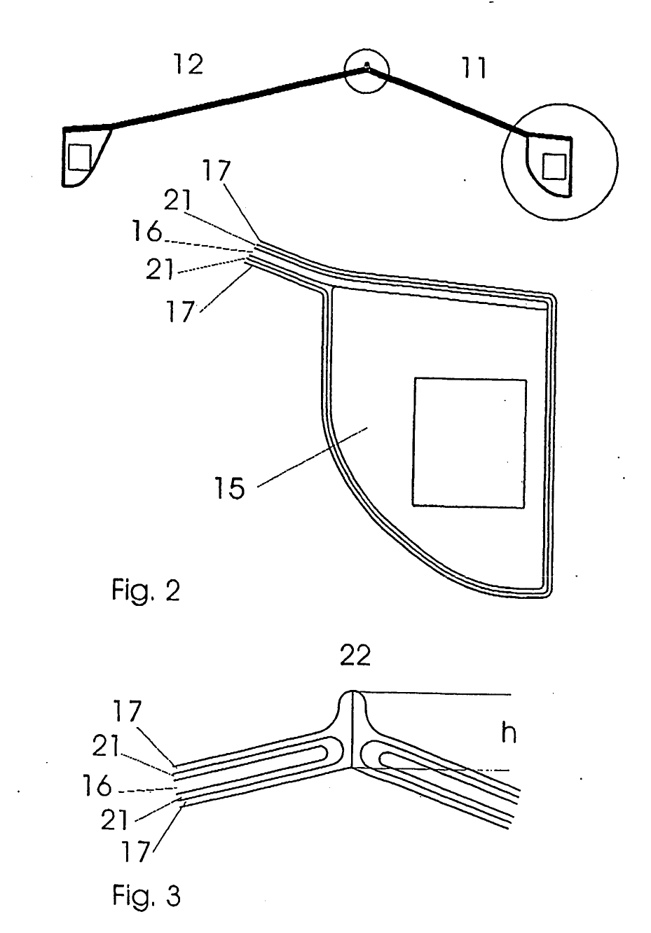

FIG. 2 is a sectional elevation along the line A-A in FIG. 1; and

FIG. 3 is a sectional elevation through the cusps 11 in the closed state.

Mitral cardiac valves are basically known with respect to their configurations from the state of the art and thus for example from WO 97/49355 or WO 97/49356. The mitral valves are comprised unitarily form a support housing 10 with a base ring carrying posts 18 substantially extending in the axial direction of the ring and whose free ends 20 from internal seats for th cusps 11 and 12 being affixed to arcuate walls serving to connect the posts 18.

The base ring comprises in plan view a closed nonround form with a common longitudinal axis but two unequal half traverse axes, whereby the posts 18, 19 lie along the longitudinal axis and form the transition locations from the one to the other half shape.

The wall 13 with the smaller curvature supports, at a greater inclination angle to the base ring base surface, a smaller area cusp 11 than the wall 14 with larger curvature.

The construction of the support housing and the cusp can be noted from FIGS. 2 and 3. From these it is clear that the cusps 11 and 12 each have a core 16 of a material with a greater hardness and a lesser bending yield strength [bending tensile strength] than those of the surface layers 17. Between these layers other additional layers 21 can be disposed with which, as is apparent from FIG. 2, also the walls 15 of the support housing 10 can be coated.

On the ends, at which the cusps 11 and 12 contact each other from opposite sides, the cusps are thickened to sealing lips 22 of the softer material 17, whereby the respective cores 16 of the cusps terminate before the sealing lips 22. The height h over which the sealing lips lie against one another on closing of the cusps amounts to at least 0.35 mm, preferably up to 0.8 mm.

To make the mitral cardiac valve prosthesis, one uses an immersion form which is comprised of two polished surfaces corresponding in shape to the cusp shape. This immersion form is coated in a plurality of immersion steps initially with a relatively soft polyurethane up to a desired thickness of the coating 17. Optionally in further immersion steps an additional intermediate layer 21 can be applied, whereby the application of each next layer can be effected in a thin laminar configuration so that a (quasi) continuous hardness gradient with each successive laminar layer can be established. Then the immersion form with the coating 17 and optionally the coating 21 is brought into a mold in which, by means of injection molding techniques, the support body is formed midway of the wall 15. In further immersion steps the cusp core 16 as well as the both layers 21 and 17 can be applied as can be deduced from FIG. 2 so that a unitary support body with cusps 11 and 12 formed thereon can be obtained.

The surface layers 17, 21 or 17 can be formed exclusively in the region of the cusps 11, 12 or also additionally over the support body 10. The cusps 11 and 12, with each of their layers 16, 17, 21 and optionally the support body 10 with the walls 15 can be comprised of polyurethane. To the extent that the embodiment shown in FIG. 2 is selected, the support body 15 also can consist of polyurethane coated polyamide.

As has already been indicated above, individual layers can be obtained instead of by an immersion process, or a casting, also by a metered application of droplets on corresponding substrates. This mode of the method is advantageous especially when a cardiac valve part having different thickness distributions is to be made as is the case for example for producing sealing lips on the free edges of the cusps.

1. (currently amended) a cardiac valve prosthesis comprised of a support housing (10) with at least two cusps (11, 12), especially a mitral cardiac valve, characterized in that the cusps (11, 12) and/or the support housing (10) have a core (15, 16) and a surface coating (17, 21) surrounding this core, whereby the core material has a greater hardness and/or a lesser bending yield strength than the surface coating.

2. (currently amended) The cardiac valve prosthesis according to claim 1 characterized in that the hardness and/or the bending yield strength of the support housing (10) and/or in the cusps (11, 12) gradually changes with increasing penetration depth from outwardly regions to inwardly lying core regions, whereby preferably the outwardly lying surface coating (17) has a modulus of elasticity of 4 N/mm2, the core material (16) has a modulus of elasticity of 40 N/mm2 to 200 N/mm2 and/or the stent material has an elasticity modulus of 200 N/mm2 to 1000 N/mm2.

3. (currently amended) The cardiac valve prosthesis according to one of claims 1 or 2 characterized in that in the cusps the core region has a thickness of 0.05 to 0.15 mm and the surface coating has a thickness of 0.02 to 0.1 mm whereby the total thickness preferably amounts to 0.2 to 0.25 mm.

4. (currently amended) The cardiac valve prosthesis according to one of claims 1 to 3 characterized in that in the cusp edge zones which come into abutment with one another upon closing of the cusps (11, 12) are configured as sealing lips (22) with an edge side thickening of a material of the surface coating, whereby the opposite side contacting surfaces-considered in the throughflow direction, have a height h of at least 0.35 mm, preferably 0.5 mm to 0.8 mm.

5. (currently amended) The cardiac valve prosthesis according to one of claims 1 to 4 characterized in that the support housing (10) and the cusps (11, 12) are composed of the same material, preferably polyurethane.

6. (currently amended) The cardiac valve prosthesis according to one of claims 1 to 5 characterized in that the support housing (10) which is preferably composed of polyurethane, is reinforced in the region of a base ring with an inlaid ring of titanium or a titanium alloy.

7. (currently amended) The cardiac valve prosthesis according to one of claims 1 to 6 characterized in that the core (15) of the support housing (10) has a greater hardness and/or reduced bending yield strength than the core (16) of the cusps (11, 12).

8. (currently amended) A method of making a cardiac valve according to one of claims 1 to 7 whereby the cusps (11, 12) are fabricated by means of an immersion process and the support body (10) by means of injection molding, characterized in that the inner surface layers 17, 21 of the cusps (11, 12) and the support body (10) are formed by at least one immersion process in a liquid solution as a unit and then by injection molding a support body core is cast thereon after which in further immersion steps the cusp core region (16) is formed thereon and finally by at least a further immersion process the outer surface layers (21, 17) of the cusps (11, 12) and the support body (10) are formed and the thus formed body is removed from the immersion mold.

9. (currently amended) The method for making a cardiac valve according to one of claims 1 to 8 characterized in that at least one of the layers (17, 21) or a core layer (15, 16) is produced by applying individual droplets of a polymer solution or droplets of a viscus polymerizable multicomponent system pointwise, in a linearly shaped row, in a worm shaped or surface application on the base body or a carrier tool, the applied material is dried and the application of the droplets and subsequent drying are repeated as often as required until the desired correctly shaped three dimensional structure or layer are formed.
